(19) United States
(12) Patent Application Publication
(10) Pub. No.: US 2012/0322025 A1
(43) Pub. Date: Dec. 20, 2012

(54) METHOD FOR FORMING DENTAL COATING AND DENTAL CAD/CAM DEVICE

(75) Inventors: Nobuyoshi Ozawa, Aichi (JP); Yasunori Sumi, Aichi (JP)

(73) Assignee: JAPAN HEALTH SCIENCES FOUNDATION, Tokyo (JP)

(21) Appl. No.: 13/579,274

(22) PCT Filed: Jun. 17, 2011
(86) PCT No.: PCT/JP2011/000835

§ 371 Date: Aug. 15, 2012

(30) Foreign Application Priority Data
Feb. 16, 2010 (JP) 2010-031742

(51) Int. Cl. A61B 6/14; A61C 5/10; A61C 13/003; A61C 5/00

(52) U.S. Cl. 433/29; 433/217.1; 433/181

(57) ABSTRACT

A dental CAD/CAM device capable of accurately forming a dental coating is provided. The device includes: an intraoral-site measurement section 100 configured to measure 3D shape data on an intraoral site 130 with an OCT probe 150 for obtaining a tomogram of an object using near-ultraviolet light; a treatment-target-tooth 3D shape data acquisition section 200 configured to acquire shape data of a treatment target tooth from 3D shape data obtained by the intraoral-site measurement section 100; and a coating object 3D shape data creation section 300 configured to create 3D shape data on a dental coating such that the dental coating matches the 3D shape data of the treatment target tooth obtained by the treatment target tooth 3D shape data acquisition section 200.

### TECHNICAL FIELD

The present disclosure relates to methods for accurately forming dental coatings and dental CAD/CAM devices.

### BACKGROUND ART

In dental clinical treatments, dental treatment is performed by filling cavities in teeth with restorative materials such as inlays or onlays, or prosthesis materials such as crowns, bridges, or implants. To obtain morphological information on a cavity in a tooth or morphological information on the inside of a mouth, for example, an indirect model of teeth or the inside of a mouth is formed using a dental impression material by a lost-wax process or other processes. Using this indirect model as a mold, a restorative material or a prosthesis material is formed.

Dental restorative/prosthesis materials (dental coatings) are conventionally formed with so-called CAD/CAM devices using computer-aided design (CAD), i.e., design with computers, and computer-aided manufacturing (CAM) of cutting out prosthesis materials by computer control. Instead of cutting, a technique of forming a three-dimensional (3D) shape by laminating materials is also widely employed to form dental restorative/prosthesis materials.

A dental restorative/prosthesis material is formed using a material such as liquid, paste, or powder is performed with a CAD/CAM system for machining from a material disk or a material block or a 3D construction by computer control in the following manner. Specifically, first, a dental impression (e.g., the shape and arrangement of teeth) of the inside of a patient's mouth including an abutment tooth in a site for which a dental restorative/prosthesis material is to be formed using a dental impression material is obtained. Based on this dental impression, a model is formed. Then, with a laser length measuring device, for example, 3D coordinate information on the shape of teeth in a site for which a dental restorative/prosthesis material is to be applied and the shape of antagonists is obtained. Based on the obtained measurement data, a dental restorative/prosthesis material is designed.

In measuring the model or the dental impression material, however, colors and surface conditions of the model or the dental impression material adversely affect the measurement accuracy in some cases. In the case of taking the dental impression with, for example, a dental impression material to form a model, it takes time to form the model.

On the other hand, Patent Document 1, for example, describes a method for 3D measurement of the inside of a mouth of a patient with X-ray equipment in order to reduce the time necessary for formation. This method takes the dental impression of teeth and jaw of a patient using a dental impression material. Then, this dental impression is scanned with an X-ray CT scanner, thereby creating 3D shape data. From this 3D shape data, 3D shape data corresponding to the surface of teeth and jaw of the patient is taken. This method is an indirect method (an extraoral method). However, general X-ray equipment displays a transmissive image. Accordingly, it is difficult to accurately measure the internal structure of a target object. The high price of X-ray equipment also inhibits widespread use thereof.

In addition, Patent Document 2, for example, describes a method for intraoral measurement of a 3D shape with one or more intraoral cameras. However, this method directly measures the 3D shape in a mouth, and thus, has low accuracy. It is particularly difficult to obtain shape data on a portion below the gingival margin to which no light reaches. In the case of forming a restorative/prosthesis material with enhanced-aesthetics, a boundary (a margin line) between natural teeth and the restorative/prosthesis material is often formed below the gingival margin. Even in the case of forming such a margin line, gingival forms a shadow, and thus, gingival retraction is needed in measurement. This necessity involves a heavy burden on patients. It is difficult to obtain information below the gingival margin in the case of a dental prosthesis material such as a crown. Accordingly, it is difficult to form a dental restorative/prosthesis material with a high accuracy of fitness based on 3D shape data obtained from an image only of the surface of intraoral tissues. In addition, since diffuse reflection and transmission occur on the tooth surface, measurement with intraoral cameras is not accurate. Accordingly, to reduce reflected light, white power or the like is sprayed onto teeth to ensure accuracy.

Dental caries of teeth and periodontal diseases are mainly caused by the influence of intraoral bacteria. Thus, formation of dental plaque of biofilm by intraoral bacteria needs to be reduced. Conventional morphology measurements cannot accurately morphologically measure the margin between a dental restorative/prosthesis material and a target teeth of treatment. These measurements insufficiently reduce secondary dental caries, periodontal diseases, fractures, cracks, etc.

### CITATION LIST

### Patent Document

PATENT DOCUMENT 1: Japanese Patent Publication No. 2007-061592
PATENT DOCUMENT 2: Japanese Translation of PCT International Application No. 2010-501278

### SUMMARY OF THE INVENTION

### Technical Problem

It is therefore an object of the present disclosure to provide a method for accurately forming a dental coating and a dental CAD/CAM device.

### Solution to the Problem

In an aspect of the present disclosure, in a method for forming a dental coating by measuring 3D shape data on an intraoral site or a dental impression of a tooth and jaw obtained from a dental impression material, creating 3D shape data on the dental coating, and forming a dental coating using the 3D shape data on the dental coating, an optical coherent tomography device is used to measure the 3D shape data on the intraoral site or the dental impression of the tooth and jaw obtained from the dental impression material.

A dental CAD/CAM device in a second aspect of the present disclosure is a dental CAD/CAM device for forming 3D shape data on a dental coating. This device includes: an intraoral-site measurement section including an OCT probe for obtaining a tomogram of an object, and configured to measure tomogram data on an intraoral site or a dental impression of a tooth and jaw obtained from a dental impression material with the OCT probe; a treatment target tooth 3D shape data acquisition section configured to acquire 3D shape data on a treatment target tooth from the tomogram data obtained by the intraoral-site measurement section; and a coating object 3D shape data creation section configured to create 3D shape data on a dental coating which matches the 3D shape data on the treatment target tooth obtained by the treatment target tooth 3D shape data acquisition section.

### Advantages of the Invention

According to the present disclosure, unlike an intraoral measurement with an X-ray CT, the internal structure of an intraoral site is three-dimensionally measured with a high resolution with an optical coherent tomography device. Accordingly, a dental coating can be accurately formed. In addition, unlike the intraoral measurement with an X-ray CT, the inside of a mouth can be directly measured with safety using no means which is harmful for human bodies. Further, even data on a shape below a gingival margin to which no light reaches can be accurately obtained with a high resolution. Accordingly, as compared to measurement using an intraoral camera, a dental coating with enhanced aesthetics can be accurately formed without the need for gingival retraction. The shapes of a dental impression material and a model can be measured without using specific powder, irrespective of color tone and surface properties of the dental impression material and the model.

Furthermore, according to the present disclosure, in consideration of conformity with alveolar bone, periodontium, cementum, and gingiva as tooth supporting tissues, a design suitable for an anatomical shape of actual teeth can be performed. Accordingly, a patient is not likely to suffer from dental caries and periodontal diseases after treatment.

Moreover, according to the present disclosure, in a margin as a outer periphery boundary between the dental coating and the treatment target tooth, a specific shape such as a chamfer shape, a shoulder shape, and a beveled shoulder shape provided to enhance treatment results can be accurately measured even below a gingival margin. Accordingly, although having been impossible with a conventional measurement of the shape of an intraoral treatment target tooth with a dental CAD/CAM, the present disclosure makes it possible to form a dental coating with an accurately equivalent to that of a lost-wax process for forming a tooth model using a dental impression material. In addition, measurement can be more accurately performed than for the shape of the inside in which the dental coating and the treatment target tooth are located. Accordingly, as compared to a dental coating formed with a conventional dental CAD/CAM technique, a dental coating exhibiting excellent compatibility and border seal properties can be formed. Thus, it is possible to reduce periodontal diseases and secondary dental caries caused by a defective dental coating.

### BRIEF DESCRIPTION OF THE DRAWINGS

FIG. 1 is a view schematically illustrating a configuration of a dental CAD/CAM device according to an embodiment.

FIG. 2 is a view schematically illustrating a configuration of an intraoral-site measurement section.

FIG. 3 is a view schematically illustrating measurement of 3D shape data on a dental impression of teeth and jaw obtained from a dental impression material with an OCT probe.

FIG. 4 is a view showing an example of a display image of 3D shape data on an intraoral site.

FIG. 5 is a diagram schematically showing a process of acquiring 3D image data on a treatment target tooth in a treatment target tooth 3D shape data acquisition section.

FIG. 6 shows an example of a 3D image of a treatment target tooth viewed in a predetermined direction.

FIG. 7 shows an example of a 3D image of a dental coating displayed on a graphic display device.

FIG. 8 is a view schematically illustrating a design of a crown shape.

FIG. 9 is a view schematically illustrating a design of an inlay shape.

FIG. 10 is a view schematically illustrating a design of a bridge shape.

### DESCRIPTION OF EMBODIMENTS

An embodiment of the present disclosure will be specifically described with reference to the attached drawings. The embodiment below is intended for easy understanding of the principle of the present disclosure. The scope of the invention is not limited to the embodiment below, and includes other embodiments expected by those skilled in the art.

Inventors of the present disclosure arrived at the invention based on the fact that accurate measurement of an intraoral site can be ensured even below the gingival margin by using optical coherent tomography (OCT). Specifically, an intraoral site or a dental impression (i.e., a model for forming a dental coating) of a tooth and jaw obtained from a dental impression material is measured with an OCT device, thereby obtaining multiple pieces of tomogram information. Based on the obtained multiple pieces of tomogram information, 3D shape data is created. Using the 3D shape data for a dental coating, a dental coating is formed.

The “dental coating” herein refers to a restorative material such as an inlay or an onlay, or prosthesis materials such as a crown, a bridge, or an implant. The “treatment target tooth” herein refers to a tooth to which the dental coating is to be applied as a dental treatment. The “treatment target tooth” herein refers to a restoration target tooth to which a restorative material is to be applied or an abutment tooth to which a prosthesis material is to be applied.

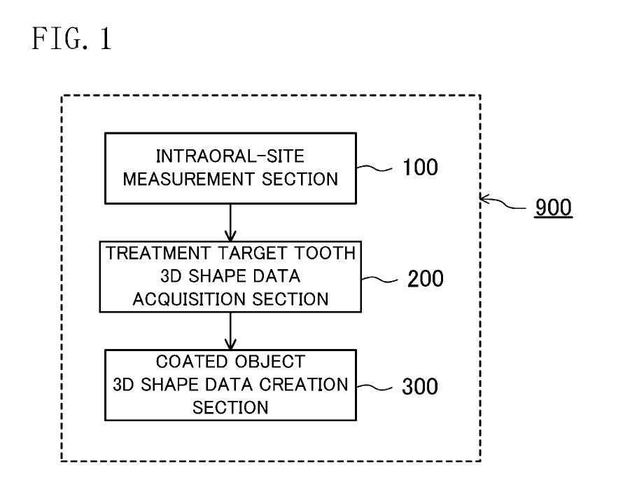

FIG. 1 is a view schematically illustrating a configuration of a dental CAD/CAM device according to this embodiment. As illustrated in FIG. 1, a dental CAD/CAM device 900 includes an intraoral-site measurement section 100, a treatment target tooth 3D shape data acquisition section 200, and a coating object 3D shape data creation section 300.

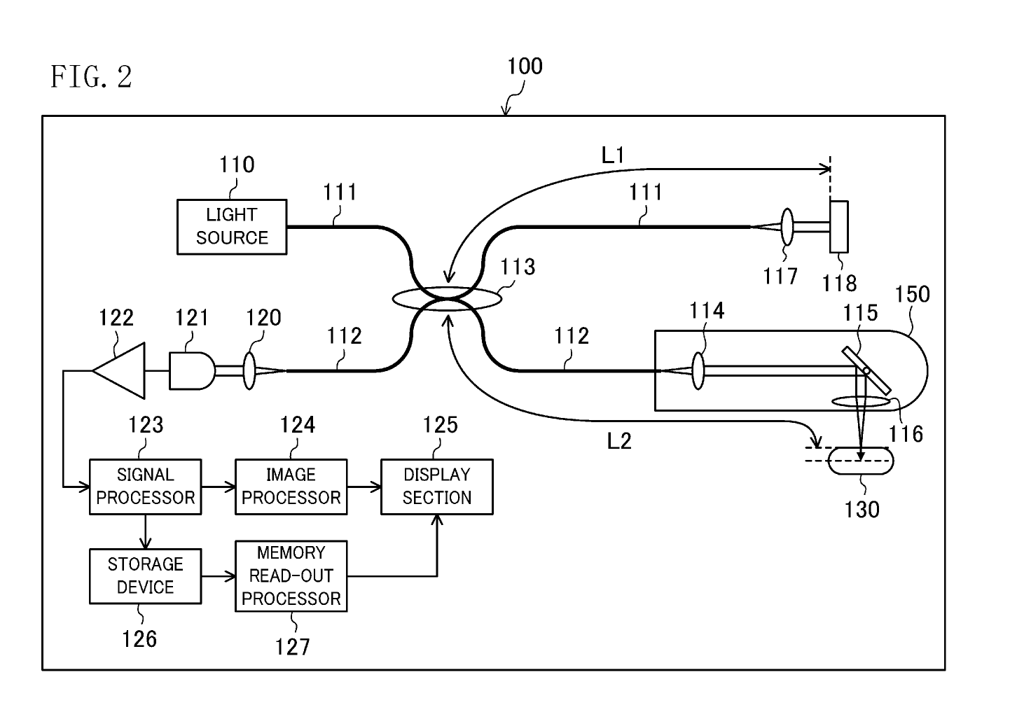

FIG. 2 is a view schematically illustrating a configuration of the intraoral-site measurement section 100. The intraoral-site measurement section 100 includes an OCT probe 150 which obtains tomograms of an object using near-ultraviolet light. This OCT probe 150 measures 3D shape data on an intraoral site 130. The intraoral site 130 is not specifically limited, and may be intraoral teeth including a tooth to be formed, the tooth surface, an occlusal surface region, or a tooth region including gingival, etc.

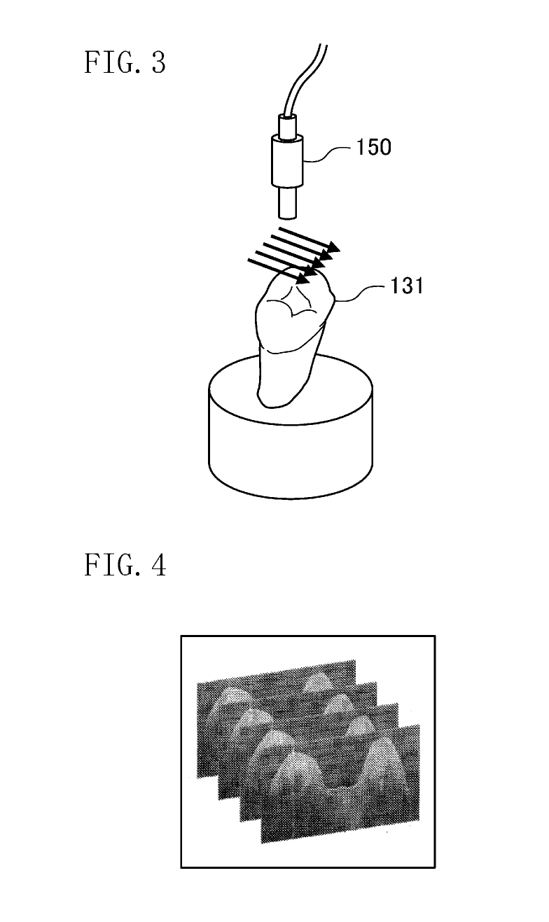

As illustrated in FIG. 3, the OCT probe 150 may measure 3D shape data on a dental impression 131 of teeth and jaw taken from a dental impression material. The impression material is not specifically limited, and may be gypsum, agar, alginate, rubber, or silicon, for example. In the manner described above, according to this embodiment, 3D shape data on an intraoral site can be not only directly measured in a mouth, but also indirectly measured outside the mouth using a model.

Referring again to FIG. 2, the intraoral-site measurement section 100 uses a light source 110 of near-ultraviolet light which emits an optical signal in a constant frequency range, as a wavelength scanning light source. Since a wavelength scanning OCT is employed, 2D data can be collected at a considerably high speed. The light source 110 has a wavelength of, for example, 700-2500 nm, which corresponds to the wavelength of near-ultraviolet light which enters the inside of an organism. The output of the light source 110 is supplied to an optical fiber 111. A coupling part 113 is provided in an intermediate portion of the optical fiber 111 by disposing another optical fiber 112 close to the optical fiber 111. An OCT probe 150 is provided at an end of the optical fiber 112. In the OCT probe 150, a collimator lens 114 changing an optical signal obtained from the light source 110 through the coupling part 113 into parallel rays and a scanning mirror 115 for scanning light are provided.

Examples of the scanning mirror 115 include a galvanometer mirror, a MEMS minor, and mirrors arranged in a round shape. The scanning minor 115 changes a reflection angle of parallel rays by rotating in a predetermined range about an axis perpendicular to the drawing sheet. Then, by rotating the scanning mirror 115, the incident position of light is changed. In this manner, tomograms as 2D information on the intraoral site 130 can be obtained. On the other hand, 3D information showing the layered structure in the intraoral site 130 can be obtained by scanning the intraoral site 130 in the direction perpendicular to the parallel rays. Although the scanning minor is constituted by a single minor in FIG. 2, it is preferable to use two scanning mirrors or to configure the scanning minor itself to be horizontally rotatable. In the case of using two scanning minors, a small amount of errors occurs in morphological information with respect to the actual size, but scanning can be performed at high speed. On the other hand, in the case of configuring the scanning mirror so that the scanning minor is capable of shifting horizontally, the scanning time somewhat increases, but errors are reduced.

An objective lens 116 is located at a position at which reflected light is received, focuses light to a site at which the intraoral site 130 is measured, and scans the site horizontally. A reference mirror 118 is provided perpendicularly to the optical axis at the other end of the optical fiber 111 with a collimator lens 117 interposed therebetween. Here, an optical distance L1 from the coupling part 113 to the reference mirror 118 is made equal to an optical distance L2 from the coupling part 113 to the surface of the intraoral site 130. The other end of the optical fiber 112 is connected to a photodetector 121 through a lens 120. The reflected light from the reference mirror 118 is light (reference light) which interferes with reflected light coming back from the intraoral site 130. The photodetector 121 is constituted by, for example, a photoreceiver and a charge coupled device (CCD) image sensor. The photodetector 121 receives reflected light from the reference mirror 118 and reflected light reflected on a measurement site, thereby obtaining a beat signal as an electrical signal. The optical fiber 111, the optical fiber 112, the coupling part 113, the collimator lens 114, the scanning mirror 115, the objective lens 116, the collimator lens 117, the reference mirror 118, and the collimator lens 120 constitute an interference optical system.

An output of the photodetector 121 is input to a signal processor 123 through an amplifier 122. The signal processor 123 performs Fourier transform on a received light signal obtained from the interference optical system, thereby obtaining a tomogram signal. An output of the signal processor 123 is supplied to an image processor 124. Based on the output from the signal processor 123, the image processor 124 acquires a 2D image of the intraoral site 130.

FIG. 4 is a view showing an example of a display image of 3D shape data on an intraoral site. A display image generated in the foregoing manner is displayed, as physically continuous sets of tomogram information, by a display section 125 as shown in FIG. 4. Information on the tomograms subjected to Fourier transform by the signal processor 123 is stored in a storage device 126.

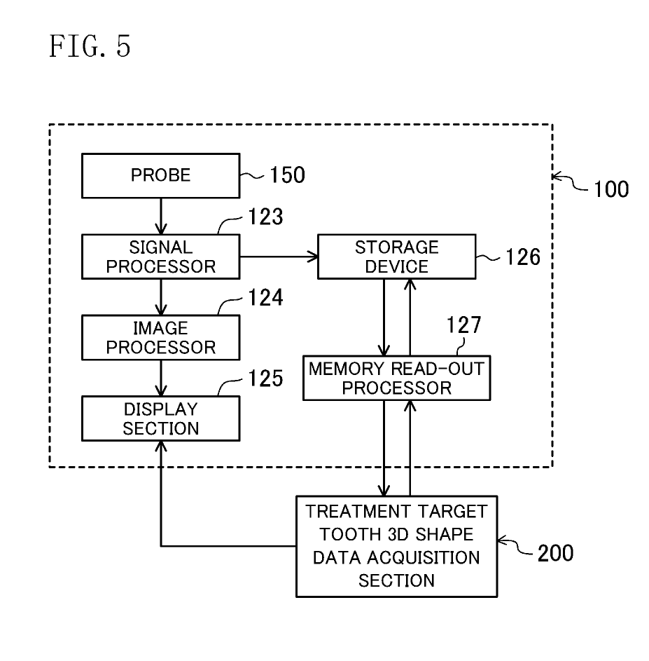

FIG. 5 is a diagram schematically showing a process of acquiring 3D image data on a treatment target tooth. As shown in FIG. 5, the treatment target tooth 3D shape data acquisition section 200 creates 3D shape data on a treatment target tooth based on a plurality of sets of tomogram information stored in the storage device 126 through a memory read-out (and write) processor 127.

The coating object 3D shape data creation section 300 includes a CAM device and a CAD device for obtaining the shape of a dental coating. Though not shown, the CAD device includes a model creator, and a program memory, for example. The CAM device receives 3D CAD data from the CAD device, and based on this 3D CAD data, creates processed data. The coating object 3D shape data creation section 300 displays a 3D image on a treatment target tooth on a graphic display device such as a display monitor of a computer, and designs the shape of a dental coating such that the shape of the dental coating conforms with the displayed treatment target tooth.

Then, use of a dental CAD/CAM device according to this embodiment will be described. For example, in a case where the dental coating is a crown, an abutment tooth is cut to be slightly tapered toward the occlusal surface, as described later. Specifically, the abutment tooth is cut to have a taper of 4° to 6°, both inclusive, relative to the perpendicular direction on the abutment tooth wall. The abutment tooth wall herein is a side wall of the abutment tooth. Then, tomograms of the abutment tooth are acquired with the tip of the OCT probe 150 shown in FIG. 2 facing toward the treatment target tooth.

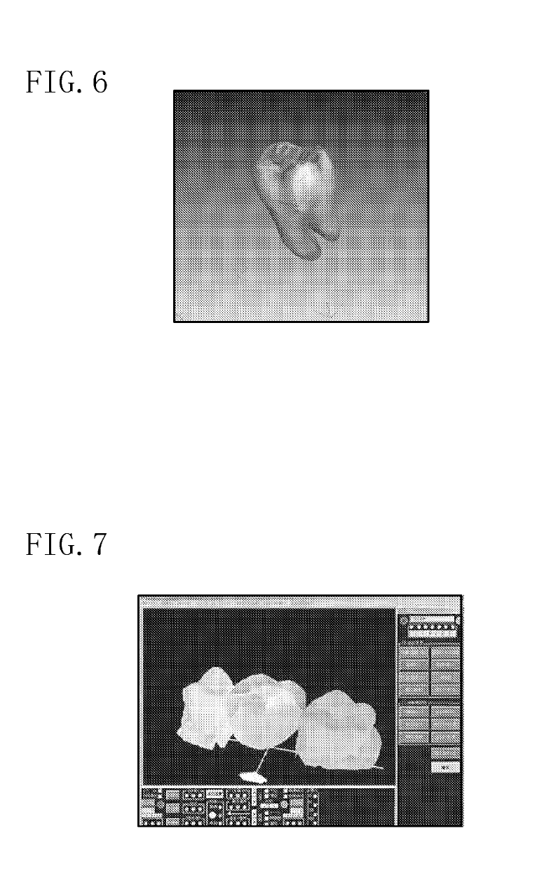

Thereafter, 3D image data on the abutment tooth is acquired. As shown in FIG. 5, a signal of light transmitted/received by the OCT probe 150 is converted into a format for display on a display monitor by the image processor 124 through the signal processor 123, and displayed on the display section 125 as a 2D image of an arbitrary tomogram of a diagnosis target site. At the same time, as shown in FIG. 4, the signal is converted into physically continuous sets of tomogram information by the signal processor 123, and stored in the storage device 126. The treatment target tooth 3D shape data acquisition section 200 creates 3D image information based on part of a plurality of sets of tomogram information recorded on a memory by the memory read-out (and write) processor 127, and records the information on the storage device 126 again. Specifically, as shown in FIG. 6, for example, tomogram information recorded on the storage device 126 is shown on the display section 125 as a 3D image viewed in a predetermined direction. The 3D shape data is not necessarily displayed on the display section 125, and the created 3D shape data may be directly transmitted or transferred to the coating object 3D shape data creation section 300 described later.

The OCT probe 150 is moved at a constant speed from a start point to an end point. A signal obtained from light transmitted from the OCT probe 150, reflected on a measurement target, and received is amplified by the signal processor 123. This signal is recorded on the storage device 126 as continuous sets of tomogram information. With the OCT probe 150 being moved at a constant speed, the physically continuous sets of tomogram information are repeatedly recorded on the storage device 126 in such a manner that tomogram information in an amount corresponding to a plurality of frames is recorded at a time. Specifically, as shown in FIG. 4, tomogram information in an amount corresponding to N frames, such as frame 1, frame 2, . . . , frame N, is recorded on the storage device along the direction in which the OCT probe 150 moves.

Subsequently, 3D image data on the dental coating is acquired. As shown in FIG. 7, for example, the coating object 3D shape data creation section 300 uses a graphic display device such as a display monitor of a computer, and designs an ideal shape of the dental coating based on the 3D image of the intraoral shape displayed on the graphic display device.

The coating object 3D shape data creation section 300 may be integrated with the intraoral-site measurement section 100 and the treatment-target-tooth 3D-shape-data acquisition section 200, or may be separated from each of the intraoral-site measurement section 100 and the treatment-target-tooth 3D-shape-data acquisition section 200. If these devices are located far from the patient and are separated from each other, for example, information such as the age, the name, intraoral photographs, and the identification number of the patient are preferably sent time in addition to measurement data on an intraoral site of the patient simultaneously with transmission of the 3D shape data.

The coating object 3D shape data creation section 300 displays a 3D image of the shape of a treatment target tooth on the graphic display device, and when necessary, also displays a 3D image of the shape of a tooth adjacent to the treatment target tooth and an antagonist. At this time, it is preferable to provide a dental coating accumulation data base in which a plurality of sets of temporary 3D shape data on a general dental coating are preferably accumulated. Specifically, it is preferable that general shape information on a target dental coating is accumulated in the dental coating accumulation data base beforehand, and data is taken out of the data base as necessary to be appropriately modified to match the shape of the treatment target tooth.

In the dental coating accumulation data base, standard shapes of human teeth are accumulated. The standard shapes of teeth may be standard shapes of portions of teeth, and shape information which differs depending on the age, the sex, etc. is preferably added. In addition, shape information on patient's teeth in healthy conditions which is previously recorded may be used. Selection of 3D shape data on a predetermined coating from the dental coating accumulation data base may be performed based on at least one of a plurality of sets of patient information including locations of a tooth, the age, and the sex. The use of the dental coating accumulation data base enables easier formation of 3D shape data on a dental coating.

A 3D image of the positional relationship between an abutment tooth and a crown is displayed on the graphic display device. The occlusion relationship between the crown and the abutment tooth figure is simulated on the display device, and the relationship with an antagonist such as a contact point is adjusted. In this manner, the shape of the crown is determined Examples of a crown whose shape can be designed include a complete veneer crown and a partial veneer crown. Examples of the partial veneer crown include a ¾ crown, a ⅘ crown, and a ⅞ crown.

Then, a design process in which a margin of the dental coating is adjusted to coincide with the margin line of the treatment target tooth and a design process for securing adhesive space in the dental coating are performed. In the dental coating whose 3D graphic is displayed on the graphic display device, the outer line of the margin of the dental coating is deformed according to the shape of the treatment target tooth, and design is performed such that the margin of the dental coating coincides with the margin line of the treatment target tooth. Thereafter, the thickness of a portion is designed with an offset for securing an adhesive layer. If the dental coating is a veneer crown, a margin line is designed below the gingival margin in consideration of aesthetics, for example. Alternatively, a margin line is designed above the gingival margin to cause enamel to remain in consideration of functionality. A portion for securing an adhesive layer is preferably located above the margin by about 0.2-2 mm in general.

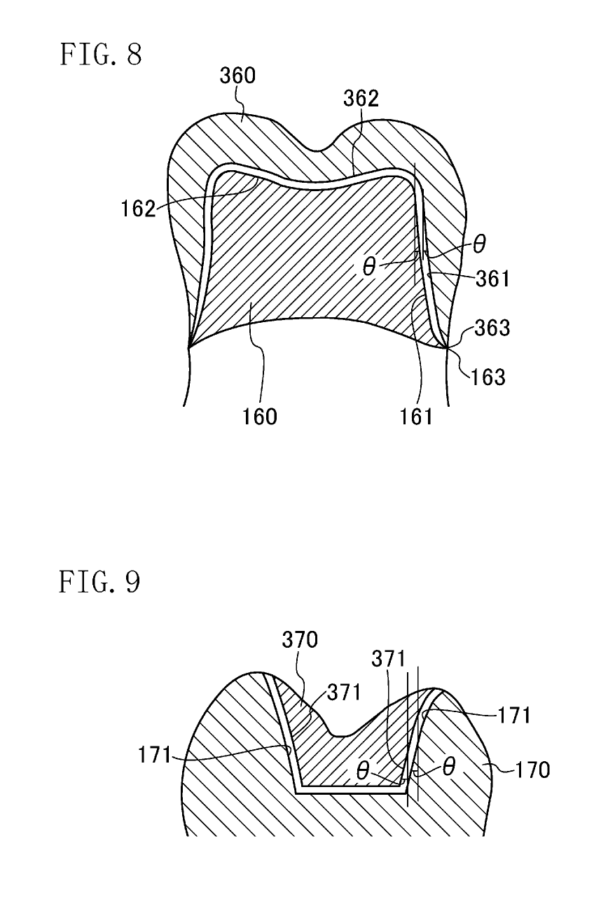

Specifically, the coating object 3D shape data creation section 300 creates 3D shape data on the dental coating by offsetting the shape of the dental coating such that that space for providing an adhesive layer with a uniform thickness is provided between the treatment target tooth and the dental coating. FIG. 8 is a view schematically illustrating a design of a crown shape, for example. The curved shape of an inner surface 362 of a crown 360 is the same as the curved shape of a surface 162 of an abutment tooth 160 facing the inner surface 362. First, the inner surface 362 of the crown 360 and the surface 162 of the abutment tooth 160 are displayed with these surfaces 362 and 162 overlapping each other, and then the inner surface 362 of the crown 360 is offset. The amount of the offset, i.e., space between the inner surface of the dental coating and the associated surface of the abutment tooth, is not specifically limited. However, smaller space is considered to have higher compatibility. The space needs to be 50 nm or less, for example, and is preferably 35 nm, more preferably 25 nm, and much more preferably 10 nm. It should be noted that the margin line 363 of the crown 360 and the margin line 163 of the abutment tooth 160 need to coincide with each other. That is, the margin line 363 of the crown 360 is not offset.

The curved shape of the inner surface 362 of the crown 360 can be formed by, for example, bonding curves expressed by surface functions such as Bezier functions together. The surface function has a plurality of control points, and locations of these control points can be displayed on the graphic display device. The curved shape of the inner surface 362 of the crown 360 changes depending on the locations of the control points. The locations of the control points are changed to change the curved shape of the inner surface 362 of the crown 360, thereby offsetting the inner surface 362 of the crown 360. The amount of movement of the control points according to the offset amount can be calculated by an offset technique of a known free-form surface. The offset process is performed through adjustment with a dial by observing the graphic display device or by inputting the amount of offset with a keyboard, for example.

As illustrated in FIG. 8, for an inner wall 361 of the crown 360 associated with an abutment tooth wall 161 of the abutment tooth 160, 3D shape data on the crown 360 is created such that the abutment tooth wall 161 is tapered at an angle θ of 4° to 6°, both inclusive, relative to the perpendicular direction. In this embodiment, the taper of the inner wall 361 is not formed by a technician based on experience, and can be formed accurately. The reasons why each of the abutment tooth wall 161 of the abutment tooth 160 and the inner wall 361 of the crown 360 is tapered at a predetermined angle are that the tapered shape allows the crown to be easily fitted in the abutment tooth and that the crown is difficult to be removed from the abutment tooth by occlusion power after being fitted into the abutment tooth. The tapered angle θ of the inner wall 361 of the crown 360 is not limited to the range from 4° to 6°, both inclusive, relative to the perpendicular direction, and may be appropriately designed depending on, for example, conditions of caries, e.g., may be in the range from 4° to 10°, both inclusive, for example.

In a case where the dental coating is an inlay, the shape is determined in the same manner. Specifically, the positional relationship between the restoration target tooth and the inlay is displayed as a 3D image on the graphic display device, the occlusion relationship with the restoration target tooth is simulated on the graphic display device to adjust the relationship with an antagonist such as a contact point, thereby determining the shape of the inlay. Then, the shape of the inlay is offset such that space for providing an adhesive layer with a uniform thickness is provided between the restoration target tooth and the inlay, thereby creating 3D shape data. Specifically, in a case where the dental coating is an inlay or an onlay, the margin line is designed to enhance the border seal property between a restorative material and a restoration target material, thereby reducing occurrence of cracks and fractures of a marginal substance of a treatment target tooth and cracks and fractures of the dental coating. In addition, iatrogenic secondary dental caries, periodontal disease, and tooth fracture due to border mismatching caused by a defective dental coating can be reduced. If a dental coating reaches an incisal margin or the occlusal surface, the dental coating is designed such that lateral force and prematurity, which are abnormal and excessive external force during occlusion, are not applied to a treatment target tooth, thereby reducing not only secondary dental caries but also occurrence of periodontal diseases, tooth fracture, and tooth cracks.

In designing the shape of an inlay, as illustrated in FIG. 9, 3D shape data on an inlay 370 is created such that an external wall 371 of the inlay 370 associated with a cavity wall 171 of a restoration target tooth 170 is tapered at an angle ranging from 4° to 6°, both inclusive, relative to the perpendicular direction. In this embodiment, the tapered shape of the external wall 371 is not formed by a technician based on experience, and can be formed accurately. The reasons why each of the cavity wall 171 of the restoration target tooth 170 and the associated external wall 371 of the inlay 370 are tapered at a predetermined angle are similar to those in the case of a crown. The taper angle θ of the external wall 371 of the inlay 370 is not limited to the angle ranging from 4° to 6°, both inclusive, relative to the perpendicular direction, and may be in the range from 4° to 10°, both inclusive. In the same manner as in the case of an inlay, in the case of an onlay in this embodiment, an offset can be provided accurately, and the external wall can be tapered.

In a case where the dental coating is a bridge, each of abutment teeth on both sides of a pontic is cut such that the resulting abutment tooth is parallel in four directions: the mesial side, the distal side, the buccal side, and the lingual side. Then, tomograms of the treatment target tooth are acquired with the tip of the OCT probe 150 facing the treatment target tooth. Then, the treatment target tooth 3D shape data acquisition section 200 acquires 3D shape data on each abutment tooth based on tomogram data obtained by the intraoral-site measurement section 100. Then, a contact point is provided at any position on the outer line of each of crowns at both sides of a tooth missing portion. After designing a prosthesis material of a tooth missing portion (a pontic) with an appropriate size, adjustment of the relationship with an antagonist and checking of a path of insertion are performed on the graphic display device. Then, the coating object 3D shape data creation section 300 creates 3D shape data on the bridge in association with 3D shape data on each abutment tooth. The 3D shape data is created by offsetting the shape of the bridge such that space for providing an adhesive layer with a uniform thickness is provided between an abutment tooth and the bridge. The use of previously registered standard data on the bridge in the dental coating accumulation data base facilitates the design. In the same manner as in the case of a crown, the margin line is designed to enhance the border seal property of the bridge to the crown abutment tooth.

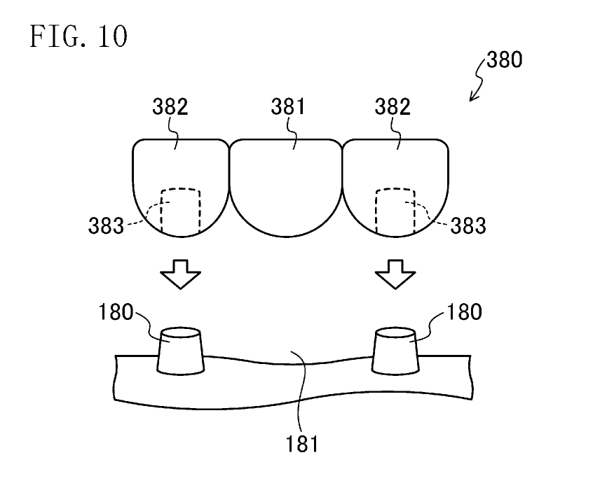

As illustrated in FIG. 10, in designing the shape of a bridge, 3D shape data on a bridge 380 is created such that a recess 383 formed in the bottom of each abutment tooth crown 382 is parallel in the four directions: the mesial side, the distal side, the buccal side, and the lingual side. An abutment tooth 180 located at each side of a tooth missing portion 181 is inserted in the recess 383 of an associated one of the abutment tooth crowns 382. In this embodiment, the parallelism of the recess 383 formed in the bottom of each abutment tooth crown 382 is not obtained by a technician based on experience, but is accurately obtained.

In this embodiment, the dental coating may include a fissure or may be deformed, if necessary. In a case where aesthetics of the dental coating is required in an anterior tooth or other teeth, the dental coating may, of course, have a thickness partially offset by, for example, forming a labial surface portion or an occlusal surface of the dental coating using a tooth crown resin, a porcelain veneer, etc.

A tool path for machining the designed shape is automatically computed, and the computed tool path is automatically computed and converted into machining data (NC data) (i.e., a so-called CAM computing). The processed data as a result of the above computing is stored as digital data in, for example, an internal storage device (e.g., a hard disk) of a computer or an external storage device of a computer.

When the 3D shape data on the dental coating is determined, 3D shape data (design data) of the final dental coating is transmitted/transferred to a machining device. The machining device may be integrated with the dental CAD/CAM device, or may be separated from a device having the function of design.

The machining device is not specifically limited as long as a 3D object can be formed from 3D shape data, and is preferably a device for forming a dental coating by machining a block- or disk-shape material or a 3D machining device using a rapid prototyping system.

The processed data is input to the machining device, is used as machining order information, and transmitted to a cutting/machining apparatus of NC control. At this same time, a block material to be used is selected and attached to automatic machining apparatus, and subjected to machining using processed data computed based on design data with a cutting tool such as a diamond bur or a carbide bur, thereby forming a dental coating.

In the case of forming a dental coating by cutting a block-shape material, in the above step of forming design data, it is necessary that the material and size, for example, of a block material to be subjected to machining is set on the graphic display device of the coating object 3D shape data creation section 300, a rest to serve as a support during machining is added on the graphic display device.

The rest corresponds to a sprue line of casting, and is displayed in a cylindrical shape in a 3D image on the graphic display device. The movement, rotation, and diameter of the rest are changed with a device such as a mouse, and the rest is set at a morphologically optimum position, avoiding an occlusal surface and a margin.

Thereafter, the size of a material set by automatic computing and the size of a dental coating to be formed are compared with each other. If the set dental coating is larger than a material to be used, the set position of the rest is changed, or the material to be used is replaced with a larger material. As described above, by defining conditions for designing a dental coating, the shape and the machining conditions, for example, finally required for cutting are determined.

In the above embodiment, among Fourier-domain OCT (FD-OCT), swept-source OCT (SS-OCT) is used. However, the present disclosure is not limited to this technique. The OCT device may employ a technique proposed by spectral-domain OCT (SD-OCT). The OCT device may also employ a technique proposed by a time-domain OCT (TD-OCT).

### INDUSTRIAL APPLICABILITY

A dental coating can be accurately formed by using an OCT device. Thus, the present disclosure is applicable to the field of dental treatment.

### DESCRIPTION OF REFERENCE CHARACTERS

100 intraoral-site measurement section
110 light source
111, 112 optical fiber
113 coupling part
114, 117 collimator lens
115 scanning minor
116 objective lens
118 reference mirror
120 lens
121 photodetector
122 amplifier
123 signal processor
124 image processor
125 display section
126 storage device
127 memory read-out processor
130 intraoral site
131 impression
150 OCT probe
200 treatment target tooth 3D shape data acquisition section
300 coating object 3D shape data creation section
360 crown
370 inlay
380 bridge
900 dental CAD/CAM device

1-15. (canceled)

16. A method for forming a dental coating by measuring 3D shape data on an intraoral site or a dental impression of a tooth and jaw obtained from a dental impression material with an optical coherent tomography device, creating 3D shape data on a treatment target tooth from an obtained tomogram, creating 3D shape data on the dental coating corresponding to 3D shape data on the treatment target tooth from the 3D shape data on the treatment target tooth, and forming the dental coating using the 3D shape data on the dental coating, wherein
the 3D shape data of the dental coating is created by offsetting a shape of the dental coating such that space for providing an adhesive layer with a uniform thickness is provided between the treatment target tooth and the dental coating over an entire surface of the dental coating associated with the treatment target tooth except for a margin line of the dental coating.

17. The method of claim 16, wherein
the dental coating is an inlay, an onlay, or a crown, and
the 3D shape data on the dental coating is formed such that an external wall of the inlay or the onlay corresponding to a cavity wall of a restoration target tooth or an inner wall of the crown corresponding to an abutment tooth wall of an abutment tooth is tapered at an angle of 4° to 6°, both inclusive, relative to a direction perpendicular thereto.

18. The method of claim 16, wherein
the dental coating is a bridge, and
the 3D shape data on the dental coating is formed such that a recess which is formed in a bottom of an abutment tooth crown located at each side of a pontic and into which an associated one of abutment teeth located at both sides of a tooth missing portion is inserted, is parallel in four directions of a mesial side, a distal side, a buccal side, and a lingual side.

19. The method of claim 16, wherein
the dental coating is designed such that a margin of the dental coating coincides with a margin line of a treatment target tooth in order to reduce occurrence of iatrogenic secondary dental caries, a periodontal disease, and tooth fracture, and an actual anatomical shape is taken into consideration by forming a contour along an anatomical shape of actual teeth and matching the dental coating and a tooth supporting tissue.

20. A dental CAD/CAM device for forming 3D shape data on a dental coating, the device comprising:
an intraoral-site measurement unit including an OCT probe for obtaining a tomogram of an object, and configured to measure tomogram data on an intraoral site or a dental impression of a tooth and jaw obtained from a dental impression material with the OCT probe;
a treatment target tooth 3D shape data acquisition section configured to acquire 3D shape data on a treatment target tooth from the tomogram data obtained by the intraoral-site measurement unit; and
a coating object 3D shape data creation section configured to create 3D shape data on a dental coating which matches the 3D shape data on the treatment target tooth obtained by the treatment target tooth 3D shape data acquisition section, wherein
the coating object 3D shape data creation section offsets a shape of the dental coating to create 3D shape data on the dental coating such that space for providing an adhesive layer with a uniform thickness is provided between the treatment target tooth and the dental coating over an entire surface of the dental coating associated with the treatment target tooth except for a margin line of the dental coating.

21. The dental CAD/CAM device of claim 20, further comprising
a dental coating accumulation data base configured to accumulate a plurality of sets of 3D shape data on a general dental coating, wherein
the coating object 3D shape data creation section selects 3D shape data on a predetermined object to be coated from the dental coating accumulation data base, and matches the selected 3D shape data with the 3D shape data on the treatment target tooth.

22. The dental CAD/CAM device of claim 20, wherein
the dental coating is an inlay, an onlay, or a crown, and
based on an occlusion relationship between a dental coating and a treatment target tooth, the coating object 3D shape data creation section creates 3D shape data on the dental coating such that the 3D shape data on the dental coating matches the 3D shape data on the treatment target tooth.

23. The dental CAD/CAM device of claim 20, wherein
the dental coating is an inlay, an onlay, or a crown, and
the coating object 3D shape data creation section creates 3D shape data on the dental coating such that an external wall of the inlay or the onlay corresponding to a cavity wall of a restoration target tooth or an inner wall of the crown corresponding to an abutment tooth wall of an abutment tooth is tapered at an angle of 4° to 6°, both inclusive, relative to a direction perpendicular thereto.

24. The dental CAD/CAM device of claim 20, wherein
the dental coating is a bridge,
the intraoral-site measurement unit measures tomogram data on an intraoral site or a dental impression of a tooth and jaw obtained from a dental impression material located at each side of a tooth missing portion,
the treatment target tooth/3D shape data acquisition section obtains 3D shape data on each abutment tooth from the tomogram data obtained by the intraoral-site measurement unit, and
the coating object 3D shape data creation section creates 3D shape data on the bridge such that the 3D shape data on the bridge matches the 3D shape data on each abutment tooth.

25. The dental CAD/CAM device of claim 20, wherein
the dental coating is a bridge, and
the coating object 3D shape data creation section creates the 3D shape data on the dental coating such that a recess which is formed in a bottom of an abutment tooth crown located at each side of a pontic and into which an associated one of abutment teeth located at both sides of a tooth missing portion is inserted, is parallel in four directions of a mesial side, a distal side, a buccal side, and a lingual side.

26. The dental CAD/CAM device of claim 20, wherein
the coating object 3D shape data creation section designs the dental coati ng such that a margin of the dental coating coincides with a margin line of a treatment target tooth in order to reduce occurrence of iatrogenic secondary dental caries, a periodontal disease, and tooth fracture, a contour is formed along an anatomical shape of actual teeth, and the dental coating and a tooth supporting tissue are matched with each other.
